(19) United States
(12) Patent Application Publication
(10) Pub. No.: US 2026/0294434 A1
(43) Pub. Date: Oct. 1, 2026

(54) COMPONENTS OF SURGICAL STAPLING INSTRUMENTS AND METHODS FOR MANUFACTURING THE SAME

(71) Applicant: INTUITIVE SURGICAL OPERATIONS, INC., Sunnyvale, CA (US)

(72) Inventors: Mark Hardyck, Belmont, CA (US); Matt Giere, San Jose, CA (US); Paul Mircik, Los Altos, CA (US); Greg Richmond, San Jose, CA (US)

(73) Assignee: INTUITIVE SURGICAL OPERATIONS, INC., Sunnyvale, CA (US)

(21) Appl. No.: 19/480,333

(22) PCT Filed: Apr. 30, 2024
(86) PCT No.: PCT/US2024/026953

§ 371 Date: Oct. 30, 2025

Related U.S. Application Data
(60) Provisional application No. 63/499,396, filed on May 1, 2023.

(51) Int. Cl. A61B 17/072; A61B 17/00; A61B 18/20

(52) U.S. Cl. CPC A61B 17/07207; A61B 18/20; A61B 2017/00526

(57) ABSTRACT

Surgical stapling instruments and components of those instruments are provided herein. Novel processes for producing such components and instruments are also provided. The components are manufactured through additive manufacturing, metal injection molding (MIM) and/or similar processes. The products are then post-processed with a laser to provide hardness and surface smoothness that meets certain performance requirements. The overall process reduces cost and lead times compared to conventional machining processes.

### CROSS-REFERENCE TO RELATED APPLICATIONS

This application claims the benefit of U.S. Provisional Application Ser. No. 63/499,396, filed May 1, 2023, the complete disclosure of which is incorporated herein by reference for all purposes.

### BACKGROUND

Minimally invasive medical techniques are intended to reduce the amount of extraneous tissue that is damaged during diagnostic or surgical procedures, thereby reducing patient recovery time, discomfort, and deleterious side effects. One effect of minimally invasive surgery, for example, is reduced post-operative hospital recovery times. The average hospital stay for a standard open surgery is typically significantly longer than the average stay for an analogous minimally invasive surgery (MIS). Thus, increased use of MIS could save millions of dollars in hospital costs each year. While many of the surgeries performed each year in the United States could potentially be performed in a minimally invasive manner, only a portion of the current surgeries uses these advantageous techniques due to limitations in minimally invasive surgical instruments and the additional surgical training involved in mastering them.

Improved surgical instruments such as tissue access, navigation, dissection and sealing instruments have enabled MIS to redefine the field of surgery. These instruments allow surgeries and diagnostic procedures to be performed with reduced trauma to the patient. A common form of minimally invasive surgery is endoscopy, and a common form of endoscopy is laparoscopy, which is minimally invasive inspection and surgery inside the abdominal cavity. In standard laparoscopic surgery, a patient's abdomen is insufflated with gas, and cannula sleeves are passed through small (approximately one-half inch or less) incisions to provide entry ports for laparoscopic instruments.

Laparoscopic surgical instruments generally include an endoscope (e.g., laparoscope) for viewing the surgical field and tools for working at the surgical site. The working tools are typically similar to those used in conventional (open) surgery, except that the working end or end effector of each tool is separated from its handle by an extension tube (also known as, e.g., an instrument shaft or a main shaft). The end effector can include, for example, a clamp, grasper, scissor, stapler, cautery tool, linear cutter, or needle holder.

Surgical clamping and cutting instruments (e.g., linear clamping, stapling, and cutting devices, also known as surgical staplers; and electrosurgical vessel sealing devices) have been employed in many different surgical procedures. For example, a surgical stapler can be used to resect a cancerous or anomalous tissue from a gastro-intestinal tract. Many known surgical clamping and cutting devices, including known surgical staplers, have opposing jaws that clamp tissue and an articulated knife to cut the clamped tissue.

Many surgical clamping and cutting instruments include an instrument shaft supporting an end effector to which a replaceable stapler cartridge is mounted. An actuation mechanism articulates the stapler cartridge to deploy staples from the stapler cartridge to staple tissue clamped between the stapler cartridge and an articulable jaw of the end effector. The articulable jaw typically includes an anvil with a staple deforming surface for providing resistance to the staples to aid in their folding. The staple deforming surface may, for example, comprise a plurality of staple pockets that are each designed to deform a corresponding staple in the stapler cartridge. Staples are pushed out of the staple cartridge through a section of tissue and against the staple pockets.

The staple pockets are typically shaped so as to receive and progressively bend or deform the legs of the staple into a closed position. The surfaces of the staple pockets, therefore, must have a substantially smooth and continuous curvature which provides a sufficiently large radii of curvature to eliminate or at least minimize tight corners that may snag or impede the staple legs that are moving along these surfaces. In addition, the staple pocket must have a sufficient hardness to adequately deform the staples. To meet these performance requirements, the anvil is typically manufactured through multiple machining processes, such as turning, milling and/or drilling, that involve cutting the anvil into the desired final shape and size by a controlled material-removal process, sometimes referred to as subtractive manufacturing.

While these manufacturing processes generally meet the performance requirements for conventional surgical stapler devices, they suffer from a number of drawbacks. For example, the multiple machining operations significantly increase the cost and length of time required to manufacture these stapler instruments. In addition, these processes require multiple machine tools that are specifically designed for an individual component of the instrument, such as an anvil. These machine tools, however, often have significant lead times which can suspend or halt manufacturing operations if the tools needed to complete the product are not available. In some instances, this leads to reduced inventory, unpredictable delivery times to customers and overreliance on demand forecasts.

Accordingly, while the surgical stapler instruments have generally proven to be effective and advantageous, still further improvements would be desirable to overcome the drawbacks with existing instruments and the processes for manufacturing those instruments. The systems, methods and devices described herein address these and other needs.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

Surgical stapling instruments and components of those instruments are provided herein. Novel processes for producing such components and instruments are also provided. In some embodiments, the products or components produced through the methods described herein may have superior performance parameters compared to products produced by conventional machining processes.

In one aspect, a surgical stapler instrument comprises at least one metallic component produced by a novel process. The process comprises initially forming the component through additive manufacturing, metal injection molding (MIM) or a similar process and then applying sufficient thermal energy to one or more surfaces of the component to shape these surfaces. This polishing or “post-processing” of the surfaces of the components increases the surface smoothness and overall hardness of these components to at least meet the same performance parameters as conventional machined parts. In addition, the overall process reduces cost and equipment lead times compared to conventional machining processes.

In one embodiment, the thermal energy is applied by emitting coherent light from a laser beam onto one or more surfaces of the component(s). Suitable lasers for use herein include carbon dioxide, gas, laser diode, fiber lasers, sold-state lasers, excimer, dye, Nd-YAD and the like. In one such embodiment, the thermal energy is applied with a fiber laser in which the active gain medium is an optical fiber doped with rare-earth elements, such as erbium, ytterbium, neodymium, dysprosium, praseodymium, thulium, holmium and combinations thereof.

In certain embodiments, the laser beam is steered to form a plurality of trajectories along one or more surfaces of the component(s). The laser may be steered through a variety of mechanisms, such as mirrors, prisms, lenses, rotating diffraction gratings, micro-mirrors, microelectromechanical systems (MEMS), Risley prisms, phased-array optics, mirror-based gimbals and the like. In one such embodiment, the fiber laser is steered with a galvanometer mechanism that rotates mirrors, such as a galvanometer scanner. The galvanometer may, for example, includes at least two mirrors (one for each axis) to direct the laser beam to any XY position in the field of view.

In embodiments, the thermal energy from the laser increases the smoothness of at least some of the surfaces of the component by a factor of about 5% to about 200%. The thermal energy may also increase the hardness of the component by a factor of about 5% to about 200%.

The components may be initially formed through metal injection molding (MIM) or similar process or an additive manufacturing process. Suitable additive manufacturing processes include stereolithography, material jetting, binding jetting, material extrusion (i.e., 3D printing), selective laser melting (SLM), selecting laser sintering (SLS), electron beam melting (EBM), direct metal laser sintering (DMLS), sheet lamination, directed energy deposition (DED) or any combination of the above. In one such embodiment, the components are formed through DLMS.

In embodiments, the surgical stapling instrument comprises an elongate shaft having a distal end effector comprising a first jaw and a second jaw movable relative to the first jaw. The instrument further includes a staple cartridge comprising a plurality of staples and an anvil configured to deform the staples. The staple cartridge may be removably positioned within one of the jaws. The components formed through the processes described herein may include the shaft, the end effector, the jaws, the staple cartridge, the anvil or certain components of these structures.

In certain embodiments, the anvil comprises a tissue engaging surface and one or more staple pockets defined in the tissue engaging surface. The one or more staple pockets comprise surfaces configured to deform the staples. These surfaces are shaped by emitting coherent light onto the one or more surfaces with a laser beam. The laser beam creates a sufficient surface smoothness to create a substantially smooth and continuous curvature in the staple pocket, thereby eliminating or minimizing tight corners that may snag or impede the staple legs that are moving along these surfaces. In addition, the laser beam increases the hardness of the anvil at the staple pockets to effectively deform the staples as they are closed onto the anvil with the movable jaw.

In embodiments, the laser beam is steered across the staple pockets by forming a series of troughs through one or more surfaces of the pockets. In an exemplary embodiment, the trajectories form a substantially hourglass shape on the one or more surfaces to increase the smoothness of the staple deforming surfaces. The hourglass shape ensures that the troughs or laser trajectories remain within the staple pockets. In certain embodiments, the areas of the anvil located immediately outside of the staple pockets are not treated with the laser to maintain a rougher surface for gripping. In other embodiments, these areas may also be treated with the laser to increase their smoothness and/or hardness.

The staple pockets may comprise first and second forming pockets aligned with each other to deform corresponding legs of a staple. In embodiments, the laser beam is preferably steered along the plurality of trajectories substantially along the direction from the first forming pocket to the second forming pocket. Applicant has discovered that steering the laser trajectories substantially “in-line” with the staple forming process provides certain benefits that include increased smoothness and hardness of the surfaces.

The laser beam is preferably steered along a plurality of trajectories to form troughs that extend from the first forming pocket to the second forming pocket. In an exemplary embodiment, each of the plurality of trajectories converges towards a longitudinal axis of the first forming pocket from a first end of the first forming pocket to a second end of the first forming pocket, and diverges away from a longitudinal axis of the second forming pocket from a first end of the second forming pocket to a second end of the second forming pocket. These trajectories are chosen to form the hourglass shape of the staple forming pockets.

The troughs formed by the trajectories may be laterally spaced from each other relative to the longitudinal axes of the first and second forming pockets. The laser may be steered along about two to 20 different trajectories, or between about five to fifteen different trajectories, preferably about 9 different trajectories.

In another embodiment, the laser beam is steered to form a plurality of layers within each of the staple pockets with each succeeding layer formed deeper within the staple pocket. The layers may be formed with each succeeding inner layer forming a deeper recess within the staple pocket such the outermost layer has the largest cross-sectional diameter and the innermost layer has the smallest cross-sectional diameter. In certain embodiments, the staple pockets are formed with about 2 to about 10 different layers, preferably about 7 layers.

In another aspect, a method for manufacturing a metallic component of a surgical stapler instrument comprises forming the component through an additive manufacturing process or a metal injection molding (MIM) process and applying sufficient thermal energy to one or more surfaces of the component to shape the one or more surfaces. The thermal energy is preferably sufficient to increase a smoothness of the one or more surfaces and/or the hardness of the component.

In embodiments, the method comprises emitting coherent light onto the one or more surfaces with a laser beam. The method may include a carbon dioxide, gas, laser diode, fiber lasers, sold-state lasers, excimer, dye, Nd-YAD laser and the like. In one such embodiment, the thermal energy is applied with a fiber laser in which the active gain medium is an optical fiber dopes with rare-earth elements, such as erbium, ytterbium, neodymium, dysprosium, praseodymium, thulium, holmium and combinations thereof.

In embodiments, the method further comprises steering the laser beam along a plurality of trajectories along the one or more surfaces of the component(s). The laser beam may be steered through a variety of mechanisms, such as mirrors, prisms, lenses, rotating diffraction gratings, micro-mirrors, microelectromechanical systems (MEMS), Risley prisms, phased-array optics, mirror-based gimbals and the like. In one such embodiment, the fiber laser is steered with a galvanometer mechanism that rotates mirrors, such as a galvanometer scanner. The galvanometer may, for example, includes at least two mirrors (one for each axis) to direct the laser beam to any XY position in the field of view.

The components may be initially formed through metal injection molding (MIM) or similar process or an additive manufacturing process. Suitable additive manufacturing processes include stereolithography, material jetting, binding jetting, material extrusion (i.e., 3D printing), selective laser melting (SLM), selecting laser sintering (SLS), electron beam melting (EBM), direct metal laser sintering (DMLS), sheet lamination, directed energy deposition (DED) or any combination of the above. In one such embodiment, the components are formed through DLMS.

### BRIEF DESCRIPTION OF THE DRAWINGS

The above and other aspects, features, and advantages of the present surgical instruments having a locking mechanism will become more apparent in light of the following detailed description when taken in conjunction with the accompanying drawings in which:

FIG. 1 is a perspective view of an illustrative surgical instrument having an end effector mounted to an elongated shaft, and an actuation mechanism;

FIG. 1A is a perspective view of illustrative surgical instrument with a robotically controlled backend mechanism;

FIG. 2 is a perspective view of the distal end portion of an illustrative surgical instrument with the jaws in the open position;

FIG. 3 is an exploded view of a cartridge configured for use with the surgical instrument of FIG. 1 including surgical fasteners, staple drivers, and a switch;

FIG. 4 is a perspective view of a stapler cartridge;

FIG. 5 is a cross-sectional view of the stapler cartridge of FIG. 4;

FIG. 6 is a perspective view of a drive member in accordance with the illustrative surgical instrument of FIG. 1;

FIG. 7 is a close-up view of staples pockets on an anvil for a surgical stapling instrument;

FIG. 8 illustrates a laser pattern for forming staple pockets;

FIG. 9 illustrates another laser pattern for forming staple pockets for an anvil of a surgical stapling instrument;

FIG. 10 illustrates one of the staple pockets of FIG. 9;

FIG. 11 is a cross-sectional side of a two-part clevis of the surgical instrument of FIG. 1;

FIG. 12 is a perspective view of the end portion of an illustrative surgical instrument with parts removed;

FIG. 13A is a cross-sectional perspective view of the actuation mechanism for a drive member in accordance with the surgical instrument of FIG. 1;

FIG. 13B is a cross-sectional side view of the actuation mechanism for a drive member in accordance with the surgical instrument of FIG. 1;

FIG. 14 illustrates a top view of an operating room employing a robotic surgical system; and

FIG. 15 illustrates a simplified side view of a robotic arm assembly.

### DETAILED DESCRIPTION

Particular embodiments of the present surgical instruments are described hereinbelow with reference to the accompanying drawings; however, it is to be understood that the disclosed embodiments are merely exemplary of the disclosure and may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure. Well-known functions or constructions are not described in detail to avoid obscuring the present disclosure in any unnecessary detail.

Surgical stapling instruments and components of those instruments are provided herein. The products are initially formed through additive manufacturing, metal injection molding (MIM) and/or similar processes that generally do not require conventional machining steps, such as turning, milling and/or drilling steps, that involve cutting the component by a controlled material-removal process, sometimes referred to as subtractive manufacturing. The products are then post-processed with a laser to provide hardness and surface smoothness that meets certain performance requirements. The overall process reduces cost and lead times compared to conventional machining processes. In some embodiments, the products produced with the methods described herein may have superior performance parameters compared products produced by conventional machining processes.

While the following description is presented with respect certain components of a surgical stapling instrument, such as a linear surgical stapler where staples are sequentially fired, it should be understood that features of the presently described surgical instruments may be readily adapted for use in any type of surgical clamping, cutting, ligating, dissecting, clipping, cauterizing, suturing and/or sealing instrument, whether or not the surgical instrument applies a fastener. For example, components produced by the processes described herein may be readily adapted for use in other types of instruments, such as linear cutting, ligating, purse-string suture applications or a combination thereof. In another example, the stapler instrument may include a circular stapler used for end-to-end and end-to-side anastomoses of certain tissues, such as the esophagus, stomach, intestines and/or rectum. In yet another example, the instrument may comprise an electrosurgical device wherein the jaws include electrodes for applying energy to tissue to treat (e.g., cauterize, ablate, fuse, or cut) the tissue.

The surgical clamping and cutting instrument may be a minimally invasive (e.g., laparoscopic) instrument or an instrument used for open surgery. Additionally, the features of the presently described surgical stapling instruments may be readily adapted for use in surgical instruments that are activated using any technique within the purview of those skilled in the art, such as, for example, manually activated surgical instruments, powered surgical instruments (e.g., electro-mechanically powered instruments), robotic surgical instruments, and the like.

The components manufactured with the processes described herein may also be incorporated into a variety of different surgical instruments, such as those described in commonly assigned, co-pending U.S. Pat. No. 10,863,988, U.S. patent application Ser. Nos. 16/966,099, 16/969,483, 16/427,427, 16/678,405, 16/904,482, 17/081,088, 17/414,714, 17/414,741, 17/787,232, 17/414,780, 17/602,272, 17/281,578, 17/414,805, 17/414,819, 17/605,230, 17/603,252, 17/147,435, 17/764,918, and International Patent Nos. PCT/US2021/65308, and PCT/US2021/65544, the complete disclosures of which are incorporated by reference herein in their entirety for all purposes as if copied and pasted herein.

Suitable additive manufacturing processes for manufacturing the components include stereolithography, material jetting, binding jetting, material extrusion (i.e., 3D printing), selective laser melting (SLM), selecting laser sintering (SLS), electron beam melting (EBM), direct metal laser sintering (DMLS), sheet lamination, directed energy deposition (DED) or any combination of the above.

In certain embodiments, the components are initially formed by DMLS, wherein a thin metal layer of powder is spread uniformly on the building plate with an automated roller. The powder bed is maintained at an elevated temperature so that the metal powder is at an optimum temperature for the sintering. The laser starts moving on a cross section of the object by heating the powder without melting it. A new layer of powder is then spread on top of the previous layer and the laser begins to form the next section. When printing is complete, the build chamber, model and excess powder inside is allowed to cool. The excess material is then recovered and recycled, leaving behind the final model.

In other embodiments, the components are initially formed metal injection molding (MIM) or a similar process, wherein a finely powdered metal is mixed with binder material to create a feedstock that is then shaped and solidified using injection molding. After molding, the part undergoes conditioning operations to remove the binder and densify the powders.

After being formed through one of the processes described above, the components are post-processed with thermal energy by emitting coherent light onto one or more surfaces of the components with a laser beam. The thermal energy is preferably sufficient to increase the smoothness of these surfaces and/or the hardness of the components to meet the performance criteria for the components in the stapler instruments. Suitable lasers for use herein include carbon dioxide, gas, laser diode, fiber lasers, sold-state lasers, excimer, dye, Nd-YAD and the like.

In one embodiment, the thermal energy is applied with a fiber laser that creates a relatively small spot size compared to, for example, a carbon dioxide laser. The active gain medium of the laser is an optical fiber doped with rare-earth elements, such as erbium, ytterbium, neodymium, dysprosium, praseodymium, thulium, holmium and combinations thereof. The fiber laser may have a wavelength of about 780 nm to about 2200 nm, preferably about 1064 nanometers The laser beam may be pulsed or continuous. The pulsed laser may, for example, have a frequency of about 10 Hz to about 10 KHz. The fiber may be single-mode or multi-mode. The laser power will vary depending on the component being manufactured.

The laser beam is steered to form a plurality of trajectories along the one or more surfaces of the component(s). The laser may be steered through a variety of mechanisms, such as mirrors, prisms, lenses, rotating diffraction gratings, micro-mirrors, microelectromechanical systems (MEMS), Risley prisms, phased-array optics, mirror-based gimbals and the like.

In one embodiment, the fiber laser is steered with a galvanometer mechanism that rotates mirrors, such as a galvanometer scanner. The galvanometer may, for example, includes at least two mirrors (one for each axis) to direct the laser beam to any XY position in the field of view.

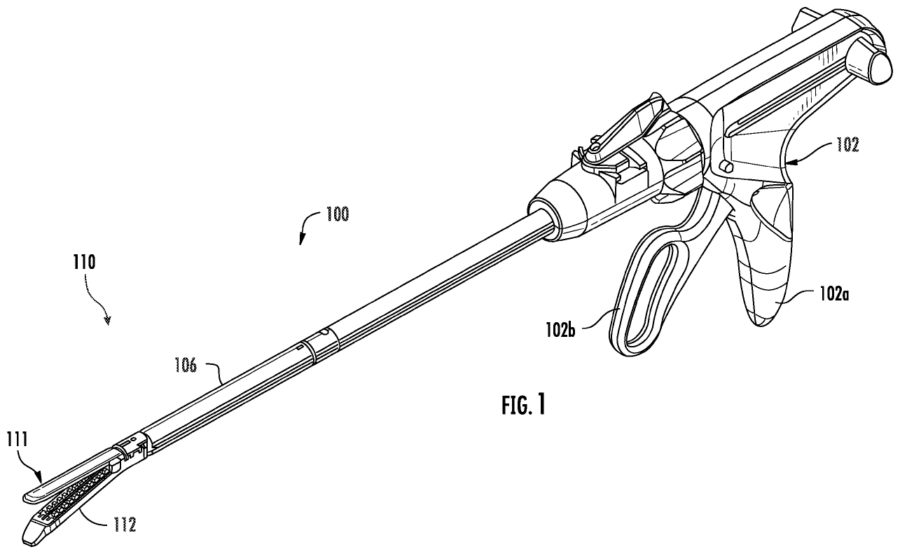

FIG. 1 is a perspective view of an illustrative surgical instrument 100 having a handle assembly 102, and an end effector 110 mounted on an elongated shaft 106. End effector 110 includes a first and second jaws 111, 112. Handle assembly 102 includes a stationary handle 102a and a moveable handle 102b which serves as an actuator for surgical instrument 100.

FIG. 1A illustrates a surgical instrument 100a that includes a backend mechanism 102c instead of the handle assembly shown in FIG. 1. Backend mechanism 102c typically provides a mechanical coupling between the drive tendons or cables of the instrument and motorized axes of the mechanical interface of a drive system. Further details of known backend mechanisms and surgical systems are described, for example, in U.S. Pat. Nos. 8,597,280, 7,048,745, and 10,016,244. Each of these patents is hereby incorporated by reference in its entirety.

The input couplers may interface with, and be driven by, corresponding output couplers (not shown) of a telesurgical surgery system, such as the system disclosed in U.S. Pub. No. 2014/0183244A1, the entire disclosure of which is incorporated by reference herein. The input couplers are drivingly coupled with one or more input members (not shown) that are disposed within the instrument shaft 106. The input members are drivingly coupled with the end effector 110. Suitable input couplers can be adapted to mate with various types of motor packs (not shown), such as the stapler-specific motor packs disclosed in U.S. Pat. No. 8,912,746, or the universal motor packs disclosed in U.S. Pat. No. 8,529,582, the disclosures of both of which are incorporated by reference herein in their entirety. Further details of known input couplers and surgical systems are described, for example, in U.S. Pat. Nos. 8,597,280, 7,048,745, and 10,016,244. Each of these patents is hereby incorporated by reference in its entirety for all purposes.

Actuation mechanisms of surgical instrument 100 may employ drive cables that are used in conjunction with a system of motors and pulleys. Powered surgical systems, including robotic surgical systems that utilize drive cables connected to a system of motors and pulleys for various functions including opening and closing of jaws, as well as for movement and actuation of end effectors are well known. Further details of known drive cable surgical systems are described, for example, in U.S. Pat. Nos. 7,666,191 and 9,050,119 both of which are hereby incorporated by reference in their entireties. While described herein with respect to an instrument configured for use with a robotic surgical system, it should be understood that the wrist assemblies described herein may be incorporated into manually actuated instruments, electro-mechanical powered instruments, or instruments actuated in any other way.

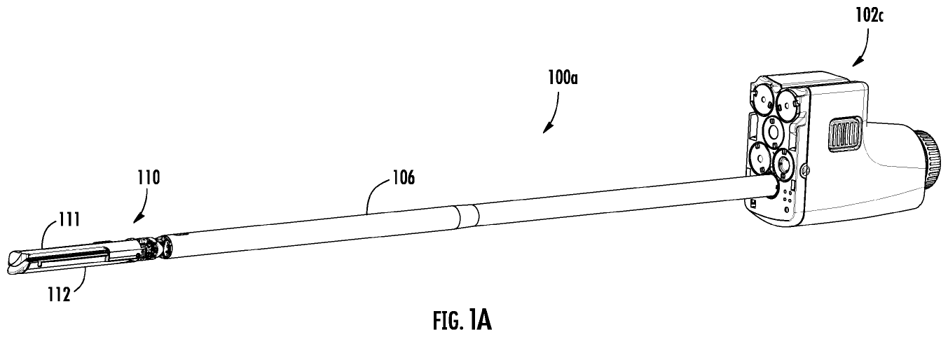

FIG. 2 shows the distal end portion of surgical instrument 100, including an end effector 110 defining a longitudinal axis X-X and having a first jaw 111, a second jaw 112, a clevis 140 for mounting jaws 111, 112 to the instrument, and an articulation mechanism, such as a wrist assembly 160. In certain embodiments, second jaw 112 is a movable jaw configured to move from an open position to a closed position relative to first jaw 111. In other embodiments, first jaw 111 is a movable jaw configured to move between open and closed positions relative to second jaw 112. In still other embodiments, both jaws 111, 112 are movable relative to each other. In the exemplary embodiment, first jaw 112 is a movable jaw 112 configured to move from an open position to a closed position relative to stationary jaw 111.

First jaw 111 includes an anvil 115 having staple-forming pockets 116. In the open position, an unused stapler cartridge 122 (sometimes referred to as a fresh or unfired reload) can be loaded into movable jaw 112 and tissue may be positioned between the jaws 111, 112. In the closed position, jaws 111, 112 cooperate to clamp tissue such that stapler cartridge 122 and the anvil 115 are in close cooperative alignment.

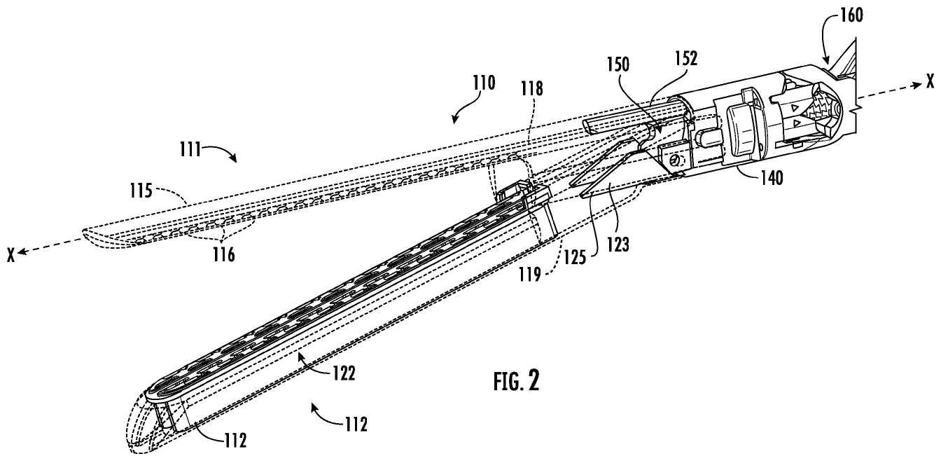

As shown in FIG. 3, stapler cartridge 122 may include a plurality of staples 124 supported on corresponding staple drivers 126 provided within respective staple retention openings or pockets 127 formed in stapler cartridge 122. In embodiments, stapler cartridge 122 further includes one or more switches 191 configured to engage a slot 196 formed on the proximal tail 195 of stapler cartridge 122. The functionality of switches 191 have been described in previously incorporated U.S. Pat. No. 10,863,988 and U.S. patent application Ser. No. 16/966,099, 16/969,483 and International Patent No. PCT/US2021/65544.

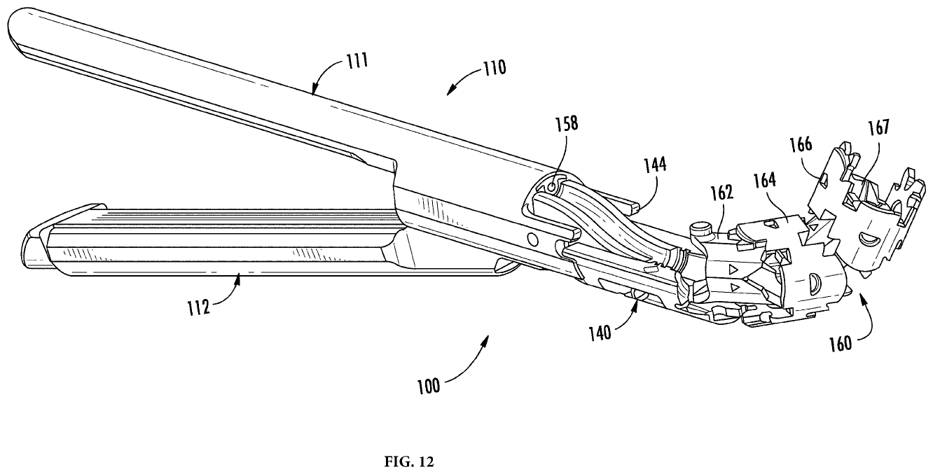

Referring again to FIG. 2, surgical instrument 100 may also include a drive member 150 configured to translate distally and retract proximally through the end effector 110. Drive member 150 may have a shuttle 123 integrally formed thereon including an inclined distal portion 125 that sequentially acts on staple drivers 126 upon distal movement of the drive member 150, camming staple drivers 126 upwardly, thereby moving staples 124 into deforming contact with anvil 115. In certain embodiments, shuttle 123 may be included within stapler cartridge 122 as a separate component. Drive member 150 includes an upper shoe 152 that is substantially aligned with and translates through a channel 118 in fixed jaw 111, while a lower shoe 154 (see FIG. 11) of drive member 150 translates through and underneath jaw 112. The details of the drive member and actuation will be described below.

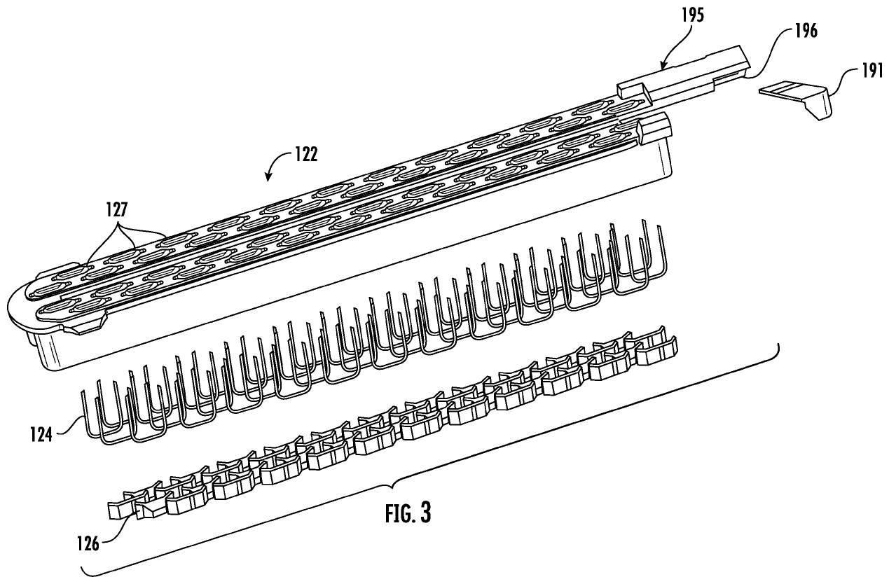

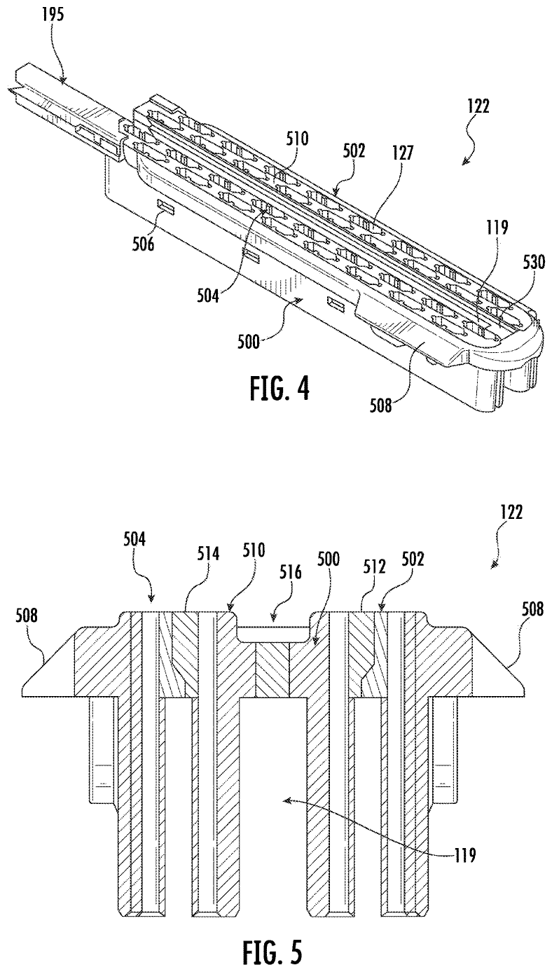

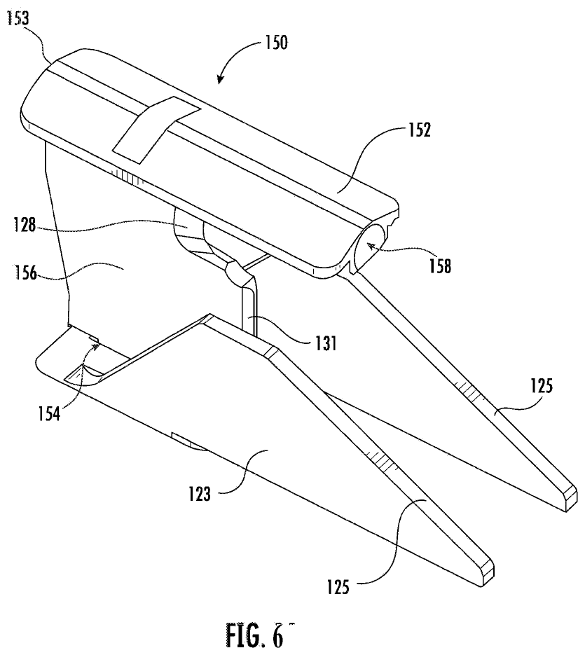

Referring now to FIGS. 4 and 5, one embodiment of a stapler cartridge 122 will now be described. As shown, cartridge 122 comprises a housing 500 having a central channel 119 for receiving drive member 150 (shown in FIG. 6 and discussed below) and first and second staple receiving assemblies 502, 504 extending longitudinally on either side of central channel 119. Each staple receiving assembly 502, 504 comprises at least one linear row of staple pockets 127 for receiving staples 124. In some embodiments, staple assemblies 502, 504 comprise two or more substantially parallel, linear rows of staple pockets 127. Cartridge 122 may further include one or more openings 506 for cooperating with detents (not shown) in second jaw 112, and one or more lateral protrusions 508 extending from a distal portion of housing 500 for cooperating with associated recesses in jaw 112.

As best shown in FIG. 5, cartridge housing 500 defines a tissue contacting surface 510 that will contact tissue when jaws 111, 112 close around the tissue. Tissue contacting surface 510 may extend laterally across housing 500 from the outside portion of staple assembly 502 to the opposite, outside portion of staple assembly 504. Tissue contacting surface 510 includes first and second lateral portions 512, 514 that generally overlie staple assemblies 502, 504 and a central portion 516 that is recessed within housing 500 relative to lateral portions 512, 514. As discussed in more detail below, drive member 150 includes a cutting element 128 (see FIG. 6) that passes through central channel 119 to dissect tissue. Simultaneously with the dissection of tissue, staples 124 are driven into the tissue on either side of the line of dissection.

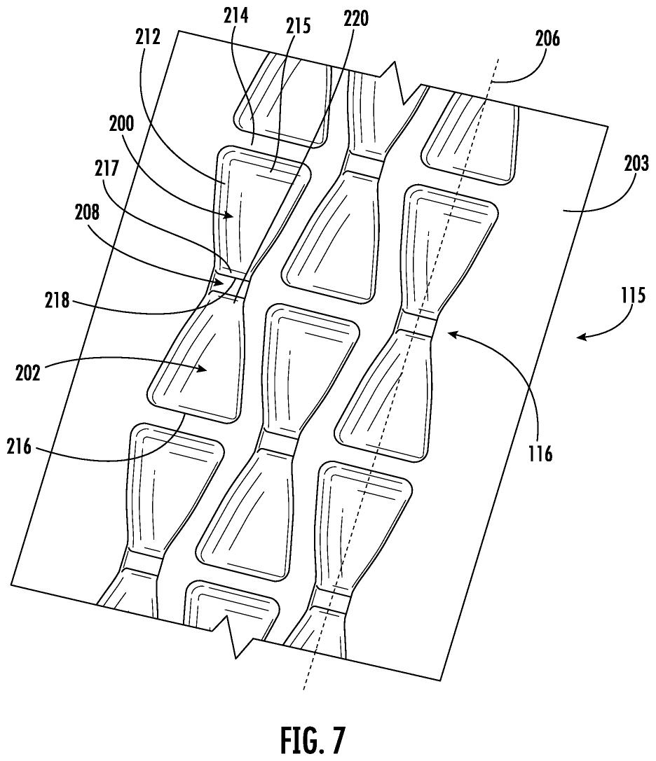

Referring now to FIG. 7, anvil 115 comprises a plurality of staple pockets 116 configured to deform staples 124 as movable jaw 112 and staple cartridge 122 are closed onto anvil 115. In the representative embodiment, anvil 116 comprises three longitudinal rows of staple pockets 116, although it will be understood that anvil 116 may comprise only one row, two rows, four rows, five rows, six rows or more. For example, in one embodiment, the staple pockets may be arranged in six longitudinal rows, with three rows of staple pockets positioned on a first side of a longitudinal axis of the anvil 115 and three rows of staple pockets positioned on a second side of the longitudinal axis.

The staple pockets 116 will have a depth, width and overall shape selected based on the desired size (e.g., diameter) and height of the staples after deformation. The staples may, for example, have open heights in the range of about 2.0 to about 4.1 mm and closed or deformed heights in the range of about 0.75 to about 2.0 mm.

In one embodiment, staple-forming pockets 116 comprise first and second forming pockets 200, 202 defined in a planar or tissue-engaging surface 203 of anvil 115. Staple forming pockets 200, 202 are aligned with each other so as to cooperate to deform the first and second legs, respectively, of each staples. Each forming pocket 200, 202 is designed to receive a leg of the staple and bend or deform the staple 124 such that the staple forms a suitable shape for securing tissue that is held between jaws 111, 112. Suitable shapes for deformed staples include B-shapes, D-shapes, M-shapes and the like.

In the representative embodiment, each staple pocket 116 includes a central longitudinal axis 206 that extends through first and second forming pockets 200, 202. A staple is intended to be deformed along the longitudinal axis 206 when deployed from a staple cartridge (i.e., the staple legs will generally deform in the direction of axis 206). The staple pockets 116 define a bridge or ridge 208 between each forming pocket 200, 202. Ridge 208 may be part of the tissue-engaging surface 203 of anvil 115, or it may be recessed slightly from this surface 203. Forming pockets 200, 202 each comprise a first outer end 214, 216 and an inner end 218, 220, respectively. Outer ends 214, 216 taper downwardly (i.e., into the depth of the pocket) in a direction substantially towards ridge 208 to provide a smooth continuous surface 215 for bending or deforming the staple legs towards ridge 208. Inner ends 218, 220 may taper downwardly substantially away from ridge 208 to provide a smooth surface 217 to receive the staple legs as they deform and prevent or at least inhibit the staple legs from snagging or otherwise being impeded from moving along these surfaces. Forming pockets 200, 202 may further include sidewalls 212 that taper downwardly towards longitudinal axis 206.

Anvil 115 is initially formed through additive manufacturing, metal injection molding (MIM) and/or similar processes described above. One or more of the surfaces of staple pockets 116 are then post-processed with a laser as described above to provide a hardness and surface smoothness that meets certain performance requirements. In one embodiment, forming pockets 200, 202 are separately shaped and post-processed by the laser. In another embodiment, forming pockets 200, 202 are post-processed simultaneously together with the laser. In either of the embodiments, ridge 208 may, or may not, be also shaped or smoothed by the laser. In certain embodiments, only portions of the forming pockets 200, 202, such as surfaces 215, 217 are shaped or post-processed by the laser.

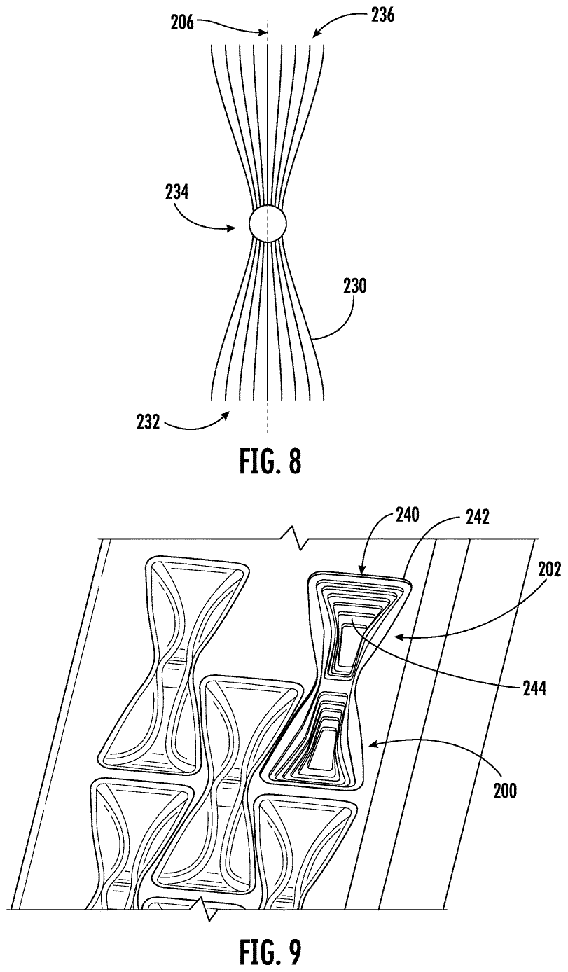

FIG. 8 illustrates one embodiment wherein the laser beam is steered across first and second forming pockets 200, 202 by forming a series of trajectories 230 through one or more surfaces of the pockets (not shown in FIG. 8). In this embodiment, the trajectories 230 of the laser may start from a first end 232 and converge towards each other as the trajectories 230 move towards the center 234 of the staple pockets (i.e., ridge 208 in FIG. 7). Trajectories 230 then diverge from each other as they advance towards a second end 236 of the pockets so as to form a substantially hourglass shape on the one or more surfaces of the pockets to increase the smoothness of staple deforming surfaces. The hourglass shape also ensures that the laser trajectories 230 remain within first and second forming pockets 200, 202. In certain embodiments, the areas of anvil 115 of surface 203 (see FIG. 7) outside of first and second forming pockets 200, 202 are not treated with the laser to maintain a rougher surface for gripping (see FIG. 7). In other embodiments, these areas may also be treated with the laser to increase their smoothness and hardness.

As shown in FIG. 8, the laser beam is preferably steered along the plurality of trajectories 230 generally along the direction of longitudinal axis 206 from first forming pocket 200 to second forming pocket 202. Applicant has discovered that steering the laser trajectories substantially “in-line” with the staple forming process provides certain benefits that include increased smoothness and hardness of the surfaces.

The trajectories 230 may be laterally spaced from each other relative to the longitudinal axes of first and second forming pockets 200, 202. The spacing between each trajectory will depend on the size of each trough and the number of trajectories. The laser may apply between about two to 20 different trajectories, or between about five to fifteen different trajectories, preferably about 9 different trajectories. The number of trajectories depends on the optical configuration of the laser and more particularly on the lateral dimension of each trough. Larger lateral dimensions generally reduce the resolution of the laser and require higher power settings. Smaller lateral dimensions increase resolution and reduce the required power settings, but require more trajectories to cover the entire span of staple forming pockets 202, 204.

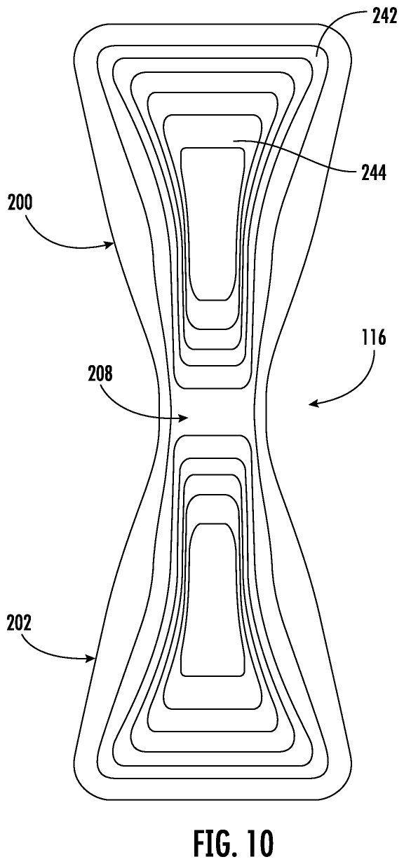

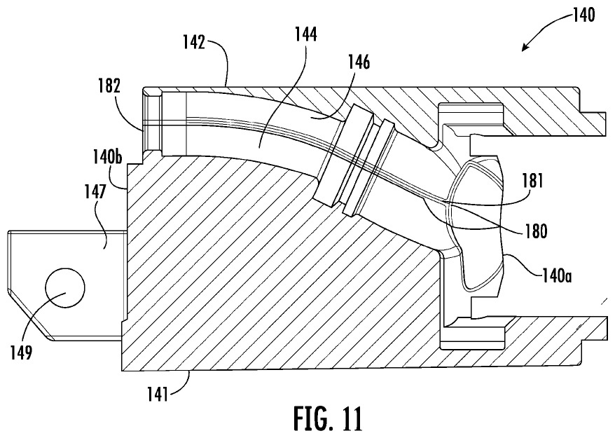

FIGS. 9 and 10 illustrate another embodiment, wherein the laser beam is steered to form a plurality of layers 240 within each of the staple forming pockets 200, 202. As shown, the layers 240 may be formed such that each succeeding layer is formed deeper into the pocket. For example, outermost layer 242 has the largest cross-sectional diameter and innermost layer 244 has the smallest cross-sectional diameter. In certain embodiments, the staple pockets are formed with about 2 to about 10 different layers, preferably about 7 layers.

In an alternative embodiment, the laser beam may be optimized to shine the hourglass light directly over the staple pockets. In this embodiment, the pockets may be post-processed without troughs or layers as described above.

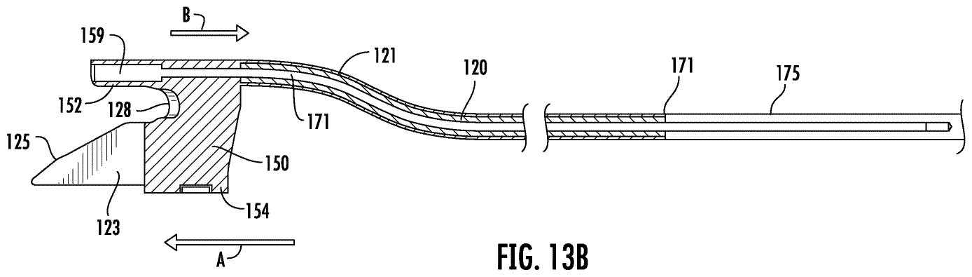

Referring now to FIG. 11, jaws 111, 112 are attached to surgical instrument 100 via clevis 140. In certain embodiments, clevis 140 includes a proximal surface 140a and a distal surface 140b. Clevis 140 further includes upper clevis portion 142 and lower clevis portion 141 that cooperate when assembled to form protrusion 145 configured to engage tabs 113 (see FIG. 13A) of jaw 111 to securely mount jaw 111 in a fixed position on instrument 100. Lower clevis portion 141 includes a pair of distally extending arms 147 for supporting movable jaw 112. Arms 147 include opening 149 for receiving a pivot pin (not shown) defining a pivot axis around which jaw 112 pivots as described in more detail below.

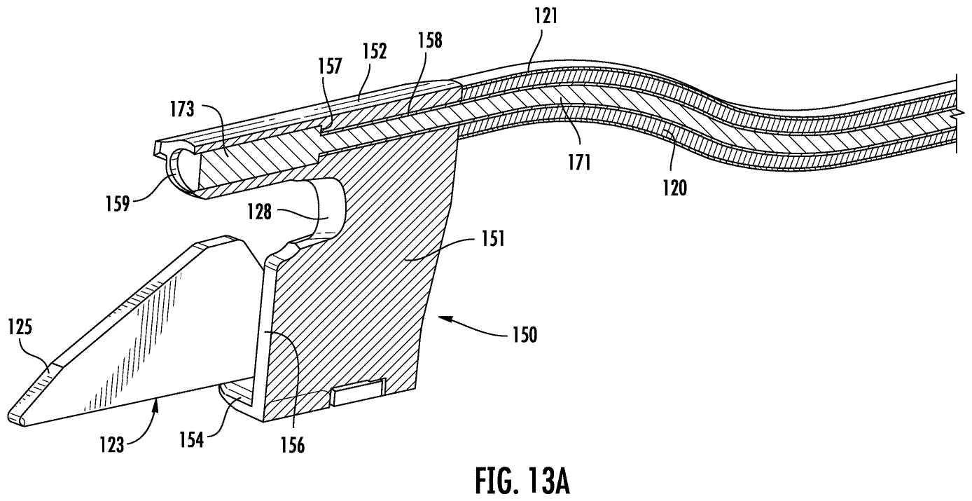

Lower clevis portion 141 also includes ramped groove 144 configured to guide a portion of an actuation coil 120 (see FIG. 13A) emerging from wrist 160 (see FIG. 12). Upper clevis portion 142 includes a complementary shaped ramped groove 146 that cooperates with ramped groove 144 of lower clevis portion 141 to form an enclosed channel 180 that guides coil 120 as it jogs upwards from wrist 160 towards distal surface 157 of upper shoe 152 of drive member 150. In embodiments, channel 180 may include a first end 181 at a central portion of proximal surface 140a and a second end 182 at a peripheral portion of distal surface 140b. In embodiments, enclosed channel 180 may be substantially “S” shaped. Although shown as a two-part clevis, it should be understood that the clevis may be a unitary structure formed, for example, by molding, machining, 3-D printing, or the like.

End effector 110 may be articulated in multiple directions by an articulation mechanism. In embodiments, the articulation mechanism may be a wrist 160 as shown, although other articulation mechanisms are contemplated. As seen in FIG. 12, wrist 160 includes a plurality of articulation joints 162, 164, 166, etc. that define a bore 167 through which an actuation mechanism (in embodiments, coil 120 and drive cable 171, see FIG. 19A) may pass. Upon exiting articulation wrist 160, coil 120 enters and passes through channel 180 of clevis 140 (see FIG. 11), ultimately engaging proximal surface 153 (FIG. 11) of upper shoe 152 of drive member 150. Other articulation mechanisms within the purview of those skilled in the art may substitute for wrist 160. One suitable articulation mechanism is described for example in U.S. Publication No. 2015/0250530, the disclosure of which is hereby incorporated by reference in its entirety.

Upon actuation of the surgical instrument, drive member 150 is advanced distally through end effector 110 to move jaws 111, 112 from the open position to the closed position, after which shuttle 123 and knife 128 are advanced distally through cartridge 122 to staple and cut tissue grasped between jaws 111, 112. Drive member 150 may be any structure capable of pushing at least one of a shuttle or a knife of a surgical stapling instrument with the necessary force to effectively sever or staple human tissue. Drive member 150 may be an I-beam, an E-beam, or any other type of drive member capable of performing similar functions. Drive member 150 is movably supported on the surgical stapling instrument 100 such that it may pass distally through cartridge 122 and upper fixed jaw 111 and lower jaw 112 when the surgical stapling instrument is fired (e.g., actuated).

As seen in FIG. 6, drive member 150 may include an upper protrusion or shoe 152, a lower protrusion or shoe 154, and a central portion 156 connecting upper and lower shoes 152, 154. Upper shoe 152 of drive member 150 is substantially aligned with and translates through channel 118 in fixed jaw 111, while lower shoe 154 of drive member 150 is substantially aligned with and translates through channel 119 and below jaw 112. Bore 158 is formed through upper shoe 152 to receive a drive cable 171 as will be described in more detail below. Proximal surface 153 of upper shoe 152 is configured to be engaged by a coil 120 of an actuation assembly such that coil 120 may apply force to upper shoe 152 to advance drive member 150 distally, i.e., in the direction of arrow “A” in FIG. 26B. A knife 128 may be formed on drive member 150 along the distal edge between upper shoe 152 and central portion 156. In embodiments, inclined distal portions 125 may be formed on either side of drive member 150.

Referring now to FIGS. 13A and 13B, an actuation assembly includes a drive cable 171, a coil 120, a sheath 121 surrounding coil 120, and a drive rod 175. Drive cable 171 includes an enlarged distal end 173. Upper shoe 152 of drive member 150 includes a bore 158 into which drive cable 171 is routed. When assembling illustrative surgical instrument 100, coil 120 and a protective sheath 121 are slipped over the free end of drive cable 171. The free end of drive cable 171 is attached to a drive rod 175 securing coil 120 and the protective sheath 121 between drive member 150 and drive rod 175 as seen in FIG. 19B. Sheath 121 may function to promote stability, smooth movement, and prevent buckling upon actuation of surgical instrument 100. Sheath 121 may be made from polyimide, or any other suitable material having the requisite strength requirements such as various reinforced plastics, a nickel titanium alloy such as NITINOL™, poly para-phenyleneterphtalamide materials such as KEVLAR™ commercially available from DuPont. Other suitable materials may be envisioned by those of skill in the art.

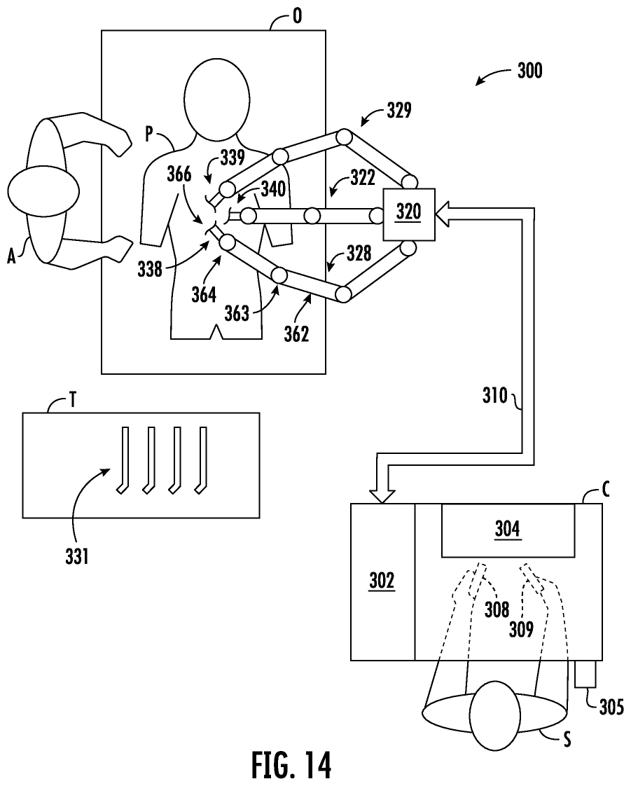

Enlarged distal end 173 of drive cable 171 resides within an enlarged distal portion 159 of bore 158 in upper shoe 152 of body 150, such that the proximal face 157 of enlarged distal end 173 may apply a retraction force on upper shoe 152 when the drive cable 171 is pulled proximally, i.e., in the direction of arrow “B” in FIG. 13B. Drive rod 175 is operationally connected to an actuator (e.g., movable handle 102b), which allows distal translation and proximal retraction of actuation assembly 190. Those skilled in the art will recognize that in a manually actuated instrument, the actuator may be a movable handle, such as moveable handle 102b shown in FIG. 1; in a powered instrument the actuator may be a button (not shown) that causes a motor to act on the drive rod; and in a robotic system, the actuator may be a control device such as the control devices described below in connection with FIG. 14. Any suitable backend actuation mechanism for driving the components of the surgical stapling instrument may be used. For additional details relating to exemplary actuation mechanisms using push/pull drive cables see, e.g., commonly owned International Application WO 2018/049217, the disclosure of which is hereby incorporated by reference in its entirety.

During actuation of illustrative surgical instrument 100, drive rod 175 applies force to coil 120, thereby causing coil 120 to apply force to upper shoe 152 of drive member 150, translating it distally (i.e., in the direction of arrow “A” in FIG. 13B) initially closing jaws 111,112 and then ejecting staples 124 from cartridge 122 to staple tissue. After stapling is complete, drive rod 175 applies a force in the proximal direction to effect retraction of drive member. During retraction, enlarged distal end 173 of drive cable 171 is obstructed by wall 157 of enlarged portion 159 of bore 158, causing drive cable 171 to apply force to upper shoe 152 of drive member 150, thereby translating drive member 150 in the proximal direction. In certain embodiments, the surgical instrument may be designed such that the drive member 150 is not retracted in the proximal direction after the staples have been fired. One of ordinary skill in the art will appreciate that drive member 150, drive cable 171, and drive rod 175 all move in unison and remain in the same relative position to each other.

In the preferred embodiment, drive cable 171 advances drive member 150 through fixed jaw 111 (instead of through the staple cartridge jaw as in conventional surgical stapling instruments). Eliminating the internal channel for the actuation mechanism from the staple cartridge provides more space in the cartridge for the staples and for the reinforcing wall discussed above. In alternative embodiments, coil 120 of actuation assembly 190 may be coupled with lower shoe 154 instead of upper shoe 152. In these embodiments, coil 120 applies force to lower shoe 154 to advance drive member 150 distally through a channel (not shown) in the lower jaw 112. In these embodiments, coil 120 will advance at least through a portion of lower jaw 112 and staple cartridge 122.

In embodiments, surgical instruments may alternatively include switches configured to be sheared along an axis, or switches having vertical cutouts designed to be engaged by an inclined distal portion of a drive member for purposes of engaging a lockout assembly, providing for reload recognition, or both, as described in International Patent Application Nos. PCT/US2019/66513 and PCT//US2019/66530, both filed on Dec. 16, 2019, the entire disclosures of which are incorporated herein by reference.

FIG. 14 illustrates, as an example, a top view of an operating room employing a robotic surgical system. The robotic surgical system in this case is a robotic surgical system 300 including a Console (“C”) utilized by a Surgeon (“S”) while performing a minimally invasive diagnostic or surgical procedure, usually with assistance from one or more Assistants (“A”), on a Patient (“P”) who is lying down on an Operating table (“O”).

The Console includes a monitor 304 for displaying an image of a surgical site to the Surgeon, left and right manipulatable control devices 308 and 309, a foot pedal 305, and a processor 302. The control devices 308 and 309 may include any one or more of a variety of input devices such as joysticks, gloves, trigger-guns, hand-operated controllers, or the like. The processor 302 may be a dedicated computer that may be integrated into the Console or positioned next to it.

The Surgeon performs a minimally invasive surgical procedure by manipulating the control devices 308 and 309 (also referred to herein as “master manipulators”) so that the processor 302 causes their respectively associated robotic arm assemblies, 328 and 329, (also referred to herein as “slave manipulators”) to manipulate their respective removably coupled surgical instruments 338 and 339 (also referred to herein as “tools”) accordingly, while the Surgeon views the surgical site in 3-D on the Console monitor 304 as it is captured by a stereoscopic endoscope 340.

Each of the tools 338 and 339, as well as the endoscope 340, may be inserted through a cannula or other tool guide (not shown) into the Patient so as to extend down to the surgical site through a corresponding minimally invasive incision such as incision 366. Each of the robotic arms is conventionally formed of links, such as link 362, which are coupled together and manipulated through motor controlled or active joints, such as joint 363.

The number of surgical tools used at one time and consequently, the number of robotic arms being used in the system 300 will generally depend on the diagnostic or surgical procedure and the space constraints within the operating room, among other factors. If it is necessary to change one or more of the tools being used during a procedure, the Assistant may remove the tool no longer being used from its robotic arm, and replace it with another tool 331 from a Tray (“T”) in the operating room.

The monitor 304 may be positioned near the Surgeon's hands so that it will display a projected image that is oriented so that the Surgeon feels that he or she is actually looking directly down onto the operating site. To that end, images of the tools 338 and 339 may appear to be located substantially where the Surgeon's hands are located.

The processor 302 performs various functions in the system 300. One function that it performs is to translate and transfer the mechanical motion of control devices 308 and 309 to their respective robotic arms 328 and 329 through control signals over bus 310 so that the Surgeon can effectively manipulate their respective tools 338 and 339. Another important function is to implement various control system processes as described herein.

Although described as a processor, it is to be appreciated that the processor 302 may be implemented in practice by any combination of hardware, software and firmware. Also, its functions as described herein may be performed by one unit, or divided up among different components, each of which may be implemented in turn by any combination of hardware, software and firmware.

For additional details on robotic surgical systems, see, e.g., commonly owned U.S. Pat. No. 6,493,608, U.S. Pat. No. 6,671, and International Application WO 2017/132611. Each of these disclosures is herein incorporated in its entirety by this reference.

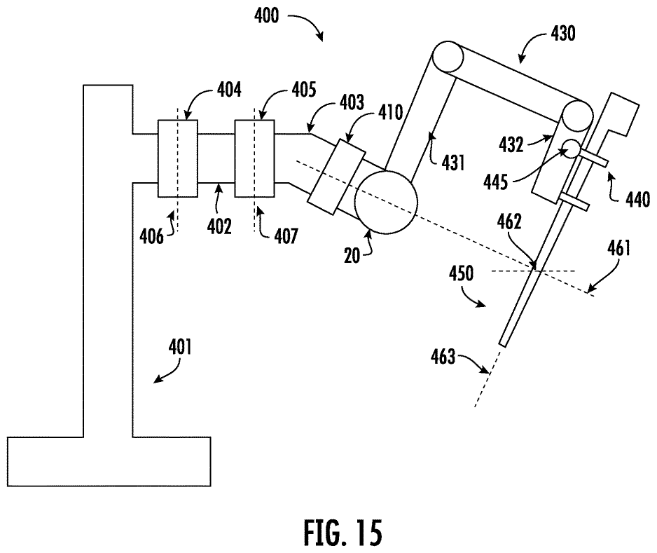

FIG. 15 illustrates, as an example, a side view of a simplified (not necessarily in proportion or complete) illustrative robotic arm assembly 400 (which is representative of robotic arm assemblies 328 and 329) holding a surgical instrument 450 (which is representative of tools 338 and 339) for performing a surgical procedure. The surgical instrument 450 is removably held in tool holder 440. The arm assembly 400 is mechanically supported by a base 401, which may be part of a patient-side movable cart or affixed to the operating table or ceiling. It includes links 402 and 403 which are coupled together and to the base 401 through setup joints 404 and 405.

The setup joints 404 and 405 in this example are passive joints that allow manual positioning of the arm 400 when their brakes are released. For example, setup joint 404 allows link 402 to be manually rotated about axis 406, and setup joint 405 allows link 403 to be manually rotated about axis 407.

Although only two links and two setup joints are shown in this example, more or less of each may be used as appropriate in this and other robotic arm assemblies described herein. For example, although setup joints 404 and 405 are useful for horizontal positioning of the arm 400, additional setup joints may be included and useful for limited vertical and angular positioning of the arm 400. For major vertical positioning of the arm 400, however, the arm 400 may also be slidably moved along the vertical axis of the base 401 and locked in position.

The robotic arm assembly 400 also includes three active joints driven by motors. A yaw joint 410 allows arm section 430 to rotate around an axis 461, and a pitch joint 420 allows arm section 430 to rotate about an axis perpendicular to that of axis 461and orthogonal to the plane of the drawing. The arm section 430 is configured so that sections 431 and 432 are always parallel to each other as the pitch joint 420 is rotated by its motor. As a consequence, the instrument 450 may be controllably moved by driving the yaw and pitch motors so as to pivot about the pivot point 462, which is generally located through manual positioning of the setup joints 404 and 405 so as to be at the point of incision into the patient. In addition, an insertion gear 445 may be coupled to a linear drive mechanism (not shown) to extend or retract the instrument 450 along its axis 463.

Although each of the yaw, pitch and insertion joints or gears, 410, 420 and 445, is controlled by an individual joint or gear controller, the three controllers are controlled by a common master/slave control system so that the robotic arm assembly 400 (also referred to herein as a “slave manipulator”) may be controlled through user (e.g., surgeon) manipulation of its associated master manipulator.

While several embodiments have been shown in the drawings, it is not intended that the disclosure be limited thereto, as it is intended that the disclosure be as broad in scope as the art will allow and that the specification be read likewise. For example, the devices disclosed herein are not limited to the mechanisms described herein for identifying and/or deactivating stapler cartridges. Other suitable devices or mechanisms are described in co-pending and co-owned International Patent Application No. PCT/US19/66513, filed Dec. 16, 2019 and entitled “SURGICAL INSTRUMENTS WITH SWITCHES FOR DEACTIVATING AND/OR IDENTIFYING STAPLER CARTRIDGES”, the complete disclosure of which is herein incorporated by reference in its entirety for all purposes. Therefore, the above description should not be construed as limiting, but merely as exemplifications of presently disclosed embodiments. Thus, the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Various alternatives and modifications can be devised by those skilled in the art without departing from the disclosure. Accordingly, the present disclosure is intended to embrace all such alternatives, modifications and variances. As well, one skilled in the art will appreciate further features and advantages of the present disclosure based on the above-described embodiments. Accordingly, the present disclosure is not to be limited by what has been particularly shown and described, except as indicated by the appended claims.

For example, in a first aspect, a first embodiment is a surgical stapler instrument comprising at least one metallic component produced by a process. The process comprises forming the component through an additive manufacturing process or a metal injection molding (MIM) process and applying sufficient thermal energy to one or more surfaces of the component to shape the one or more surfaces.

A second embodiment is the first embodiment wherein the thermal energy is sufficient to increase a smoothness of the one or more surfaces.

A third embodiment is any combination of the first 2 embodiments, wherein the thermal energy is sufficient to increase a hardness of the component.

A 4^{th} embodiment is any combination of the first 3 embodiments, wherein the smoothness is increased by a factor of at least about 5% to about 200%.

A 5^{th} embodiment is any combination of the first 4 embodiments, wherein the hardness is increased by a factor of at least about 5% to about 200%.

A 6^{th} embodiment is any combination of the first 5 embodiments, wherein the thermal energy is applied by emitting coherent light onto the one or more surfaces with a laser beam.

A 7^{th} embodiment is any combination of the first 6 embodiments, further comprising steering the laser beam along a plurality of trajectories along the one or more surfaces.

An 8^{th} embodiment is any combination of the first 7 embodiments, wherein the plurality of trajectories form a substantially hourglass shape on the one or more surfaces.

A 9^{th} embodiment is any combination of the first 8 embodiments, further comprising forming a plurality of troughs in the one or more surfaces with the laser beam.

A 10^{th} embodiment is any combination of the first 9 embodiments, wherein the component comprises a first jaw of an end effector of the instrument, the instrument comprising a second jaw for receiving a staple cartridge with a plurality of staples.

An 11^{th} embodiment is any combination of the first 10 embodiments, wherein the first jaw is an anvil comprising an inner surface configured to deform the staples.

A 12^{th} embodiment is any combination of the first 11 embodiments, wherein the inner surface of the anvil comprises a plurality of staple pockets each having a staple deforming surface facing towards the second jaw, the method comprising sufficient thermal energy to the staple deforming surfaces to increase a smoothness of said staple deforming surfaces.

A 13^{th} embodiment is any combination of the first 12 embodiments, wherein the staple deforming surfaces comprise first and second forming pockets configured to deform corresponding legs of a staple, the method further comprising steering a laser beam along a plurality of trajectories from the first forming pocket to the second forming pocket.

A 14^{th} embodiment is any combination of the first 13 embodiments, wherein each of the plurality of trajectories converges towards a longitudinal axis of the first forming pocket from a first end of the first forming pocket to a second end of the first forming pocket, and diverges away from a longitudinal axis of the second forming pocket from a first end of the second forming pocket to a second end of the second forming pocket.

A 15^{th} embodiment is any combination of the first 14 embodiments, wherein the plurality of trajectories are laterally spaced from each other relative to the longitudinal axes of the first and second forming pockets.

A 16^{th} embodiment is any combination of the first 15 embodiments, further comprising steering the laser beam through about five to about fifteen different trajectories.

A 17^{th} embodiment is any combination of the first 16 embodiments, further comprising forming first and second layers in the one or more surfaces, wherein the first layer is an outermost layer and the second layer is an innermost layer, wherein the first layer has a larger cross-sectional area than the second layer.

An 18^{th} embodiment is any combination of the first 17 embodiments, wherein the component is formed by direct metal laser sintering (DLMS).

A 19^{th} embodiment is any combination of the first 18 embodiments, wherein the component is formed by a MIM process.

1. A surgical stapler instrument comprising an end effector having a metallic first jaw produced by a process comprising:
forming an anvil on an inner surface of the first jaw through an
additive manufacturing process or a
metal injection molding (MIM) process; and
applying sufficient thermal energy to one or more surfaces of the component to shape the one or more surfaces.

2. The surgical stapler instrument of claim 1, wherein the thermal energy is sufficient to increase a smoothness of the one or more surfaces.

3. The surgical stapler instrument of claim 1, wherein the thermal energy is sufficient to increase a hardness of the component.

4. (canceled)

5. (canceled)

6. The surgical stapler instrument of claim 1, wherein the thermal energy is applied by emitting coherent light onto the one or more surfaces with a laser beam.

7. The surgical stapler instrument of claim 6, further comprising steering the laser beam along a plurality of trajectories along the one or more surfaces.

8. The surgical stapler instrument of claim 7, wherein the plurality of trajectories form a substantially hourglass shape on the one or more surfaces.

9. The surgical stapler instrument of claim 7, further comprising forming a plurality of troughs in the one or more surfaces with the laser beam.

10. The surgical stapler instrument of claim 1, wherein the instrument comprises a second jaw for receiving a staple cartridge with a plurality of staples.

11. (canceled)

12. The surgical stapler instrument of claim 1, wherein the inner surface of the anvil comprises a plurality of staple pockets each having a staple deforming surface facing towards the second jaw, the method comprising sufficient thermal energy to the staple deforming surfaces to increase a smoothness of said staple deforming surfaces.

13. The surgical stapler instrument of claim 12, wherein the staple deforming surfaces comprise first and second forming pockets configured to deform corresponding legs of a staple, the method further comprising steering a laser beam along a plurality of trajectories from the first forming pocket to the second forming pocket.

14. The surgical stapler instrument of claim 1, wherein each of the plurality of trajectories converges towards a longitudinal axis of the first forming pocket from a first end of the first forming pocket to a second end of the first forming pocket, and diverges away from a longitudinal axis of the second forming pocket from a first end of the second forming pocket to a second end of the second forming pocket.

15. (canceled)

16. (canceled)

17. (canceled)

18. (canceled)

19. (canceled)

20. A method of forming an anvil of a surgical stapler instrument, the method comprising:
forming the anvil through an additive manufacturing process or a metal injection molding (MIM) process; and
applying sufficient thermal energy to one or more surfaces of the anvil to at least one of shape or increase the smoothness or hardness of the one or more surfaces.

21. The method of claim 20, further comprising applying sufficient thermal energy to increase the smoothness or hardness of the one or more surfaces by a factor of at least about 5% to about 200%.

22. (canceled)

23. The method of claim 20, further comprising emitting coherent light onto the one or more surfaces with a laser beam.

24. The method of claim 23, further comprising steering the laser beam along a plurality of trajectories along the one or more surfaces.

25. (canceled)

26. The method of claim 24, further comprising creating a plurality of troughs in the one or more surfaces with the laser beam.

27. (canceled)

28. (canceled)

29. (canceled)

30. (canceled)

31. A surgical stapler instrument comprising:
an end effector comprising a first jaw and a second jaw movable relative to the first jaw;
a staple cartridge comprising a plurality of staples; and
an anvil configured to deform the staples, the anvil comprising a tissue engaging surface; and
one or more staple pockets defined in the tissue engaging surface, wherein the one or more staple pockets comprise surfaces configured to deform the staples, wherein said surfaces are shaped by emitting coherent light onto the one or more surfaces with a laser beam.

32. The surgical stapler instrument of claim 31, wherein the staple pockets each comprise a first forming pocket and a second forming pocket configured to deform corresponding legs of one of the staples, wherein the first and second forming pockets are shaped by steering a laser beam along a plurality of trajectories from the first forming pocket to the second forming pocket.

33. The surgical stapler instrument of claim 32, wherein each of the plurality of trajectories converges towards a longitudinal axis of the first forming pocket from a first end of the first forming pocket to a second end of the first forming pocket, and diverges away from a longitudinal axis of the second forming pocket from a first end of the second forming pocket to a second end of the second forming pocket.

34. The surgical stapler instrument of claim 33, wherein the plurality of trajectories are laterally spaced from each other relative to the longitudinal axes of the first and second forming pockets.

35. (canceled)
